(19) United States
(12) Patent Application Publication
(10) Pub. No.: US 2025/0052707 A1
(43) Pub. Date: Feb. 13, 2025

(54) MONITORING SYSTEM WITH A GAS MEASURING DEVICE

(71) Applicant: Dräger Safety AG & Co. KGaA, Lübeck (DE)

(72) Inventors: Tilman von BLUMENTHAL, Lübeck (DE); Hans-Ullrich HANSMANN, Lübeck (DE); Henning GERDER, Lübeck (DE); Robert JAHNS, Lübeck (DE); Christoph OSTERLOH, Lübeck (DE)

(21) Appl. No.: 18/798,948

(22) Filed: Aug. 9, 2024

(30) Foreign Application Priority Data
Aug. 10, 2023 (DE) 10 2023 121 409.8

(51) Int. Cl. G01N 25/18; G01N 21/3504; G01N 33/00; G01N 33/497

(52) U.S. Cl. CPC G01N 25/18; G01N 21/3504; G01N 33/004; G01N 33/497

(57) ABSTRACT

A monitoring system (100) with a gas measuring device (72) as well as to a process for operating a monitoring system (100). The monitoring system (100) is used to monitor the breathing gas supply of an airplane pilot in an aircraft. The principal components in the breathing gas (10) are formed by air (5), i.e., essentially defined quantities of oxygen, carbon dioxide, nitrogen and moisture or water vapor. On the one hand, information on the exact quantity of oxygen and carbon dioxide in the breathing gas mixture is essential to assess whether a breathing gas mixture for an airplane pilot meets specific requirements. The monitoring system (100) with the gas measuring device (72) offers possibilities for obtaining indications or estimates (44) in respect to possible contaminations by additional components in the breathing gas supply of an airplane pilot.

### CROSS REFERENCE TO RELATED APPLICATIONS

This application claims the benefit of priority under 35 U.S.C. § 119 of German Application 10 2023 121 409.8, filed Aug. 10, 2023, the entire contents of which are incorporated herein by reference.

### TECHNICAL FIELD

The present invention pertains to a monitoring system with a gas measuring device as well as to a process for operating a monitoring system. The monitoring system is used here to monitor the supply of breathing gas for an aircraft pilot in an aircraft. Airplanes or aircraft are defined here as an airplane or helicopter of civil or military aviation, e.g., passenger airplanes in scheduled or charter transport service as well as ultrafast aircraft flying close to the range or above the range of ultrasonic speeds. Especially flights with jets at ultrasonic speeds and/or at flight altitudes above 15,000 μm above sea level impose high requirements on the fitness to fly of the flight crew. The fitness to fly with physical and mental fitness, attention, ability to concentrate and alertness must be guaranteed at all times at high altitudes, during high-speed maneuvers or flight positions, for example, curved flight, nose-dives, inverted flight at high speeds and with high accelerations. In addition to the personal and health-related conditions of the aircraft pilot and a reliable outfitting of the aircraft, secured supply of the aircraft pilot with satisfactory breathing air, which is not harmful to the health, is very essential as well for a safe flight operation.

### BACKGROUND

Paramagnetic methods, which are based on the fact that oxygen molecules are paramagnetic based on their permanent magnetic dipole moments, whereas most other gases possess diamagnetic properties, are frequently used to determine an oxygen concentration in gases. It is generally known that the thermal conductivity changes in paramagnetic gases under the influence of magnetic fields. The cause of this behavior is obviously the fact that paramagnetic gases have a permanent magnetic moment, which does not, however, normally become outwardly manifest based on the thermal motion of the gas molecules. However, a sufficiently strong external magnetic field ensures that the magnetic dipole moments of the individual gas molecules are oriented. This leads, on the one hand, to a change in susceptibility, which leads to an increase of the magnetic flux, and, on the other hand, a certain molecular arrangement becomes established in the gas, as a result of which the degrees of freedom and hence the possibilities of transmitting thermal energy to adjacent molecules by shocks become limited. As a result, the thermal conductivity of the gas will change slightly.

Paramagnetic measuring devices for determining oxygen concentrations, especially also in breathing gases, are known, for example, from U.S. Pat. No. 6,952,947 B2, US 2011094293 A1, U.S. Pat. Nos. 8,596,109 B2, 9,360,441 B2, 6,895,802 B2, 6,405,578 B2, 6,430,987 B1, 4,808,921 A, 4,683,426 A, 3,646,803 A, 3,584,499 A, and 2,944,418 A.

A basic principle for measuring oxygen in a measuring chamber with the use of changes in the heat conduction in connection with paramagnetism is described in U.S. Pat. No. 6,430,987 B1. US 20230114548 A1 describes a possibility of determining the presence of an anesthetic gas in the breathing gas mixture in addition to the measurement of oxygen.

It is known that the composition of breathing gases can be measured optically. The absorption of light in a defined wavelength range specific of the particular gas is used here as the indicator of the concentration of the gas in question. Among other things, the concentrations of volatile anesthetic gases, carbon dioxide (CO₂) as well as nitrous oxide (N₂O) are measured in this manner in the field of medicine, especially during the performance of an anesthesia of a living being, in the breathing gas mixture of patients being ventilated.

An infrared optical gas measuring device is described in U.S. Pat. No. 5,739,535. An infrared optical carbon dioxide sensor, a so-called IR carbon dioxide sensor, is known from U.S. Pat. No. 8,399,839 B2. A combination sensor comprising an infrared optical carbon dioxide sensor with a flow sensor, which can be arranged in the main stream in the breathing gas path of a patient, is known from U.S. Pat. No. 6,571,622 B2. Infrared optical carbon dioxide sensors, which may be arranged in the side stream or in the breathing gas path of a patient, are known from US 2004238746 A and US 2002036266 A. U.S. Pat. Nos. 6,954,702 B, 7,606,668 B, 8,080,798 B, 7,501,630 B, 7,684,931 B, 7,432,508 B, and 7,183,552 B show gas measuring systems for detecting gas concentrations in the side stream and in the main stream.

A monitoring system for monitoring pilots and/or copilots is known from US 20210405008 A1. As part of the equipment of the aircraft pilot, the monitoring system is preferably configured as an autarchic and mobile unit carried on the body with an independent energy supply unit.

The principal components of the breathing gas mixture are formed by quantities of oxygen, carbon dioxide, nitrogen and moisture or water vapor. On the one hand, information on the exact contents of oxygen and carbon dioxide in the breathing gas mixture is essential for estimating whether a breathing gas mixture meets the requirement of an aircraft pilot. It is, however, just as essential to have estimates in respect to possible impurities in the breathing gas mixture in order to be able to estimate a possible health hazard to the aircraft pilot due to impurities present in the breathing gas mixture as early as possible during the flight operation.

### SUMMARY

It is an object of the invention to identify a state in the breathing gas supply of the aircraft pilot to determine whether foreign gas components are present in the breathing gas mixture, especially to determine whether another gas, which is different from carbon dioxide, nitrogen, moisture or water vapor and oxygen, is present.

The object is accomplished by monitoring system features and gas measuring device features according to the invention.

The object is accomplished by a monitoring system with a gas measuring device having features according to the invention as well as by a process having features according to the invention.

Further features and details of the present invention appear from this disclosure including the description, the claims and from the drawings.

Features and details that are described in connection with the monitoring system according to the present invention are, of course, also valid in connection with the process according to the present invention and also vice versa, so that reference is and can always mutually be made to the individual aspects of the present invention concerning the disclosure.

According to a first aspect of the present invention, embodiments are shown, which show a monitoring system, which has at least the following components:
a gas measuring device,
a gas transport module and
a control and analysis unit.

The monitoring system according to the present invention for monitoring a gas composition of a breathing gas supply of an aircraft pilot in airplanes or aircraft is configured with the gas measuring device, with the gas transport module and with the control and analysis unit in order to organize, check, control or regulate a process for the measurement-based monitoring of the breathing gas supply.

A memory (data storage device), which is configured to store properties of different gases or gas mixtures, is connected to the control and analysis unit. Thus, typical properties of such gases, gas mixtures or vapors, which may additionally be present as components, e.g., fumes, soot, unburnt fuel, fuels and operating materials, such as, e.g., lubricant or coolant, in addition to the gases oxygen (O₂), moisture or water vapor (H₂O), nitrogen (N₂) and carbon dioxide (CO₂) during the flight operation, can thus be stored in the memory.

The gas transport module is configured by means of a measuring line (sampling line) for feeding defined quantities of breathing gas mixture from a measurement location through a gas inlet at the monitoring system to the gas measuring device.

The gas measuring device has a moisture sensor (humidity sensor) for determining a moisture in the breathing gas mixture, which sensor is configured in combination with the control and analysis unit to determine a value that indicates a moisture or a water content in the breathing gas mixture.

The gas measuring device has, in addition, a pressure sensor for determining a pressure level in the breathing gas mixture, which sensor is configured in combination with the control and analysis unit to determine a value that indicates the pressure level of the breathing gas mixture.

The gas measuring device further has a temperature sensor for determining a temperature of the breathing gas mixture, which sensor is configured in combination with the control and analysis unit to determine a value that indicates a temperature of the breathing gas mixture.

The gas measuring device has a thermoelectric device, formed with thermocouples or thermopiles. An electromagnetic device, formed with coils and magnetically conductive materials, is arranged at the thermoelectric device. The electromagnetic device is configured, in combination with the control and analysis unit, to allow a magnetic field to act cyclically on the thermocouples or on the thermopiles, and the thermoelectric device is further configured, in combination with the control and analysis unit, to carry out a determination of a thermal conductivity of the breathing gas and to determine a value that indicates situations of the thermal conductivity as a function of an effect or without an effect of the magnetic field on the quantities of breathing gas mixture fed by means of the gas transport module. If additional gas components, especially molecules of hydrocarbons, are present in the breathing gas mixture, the heat conduction of the gas mixture as a whole changes, both under an influence of the magnetic field and without an influence of the magnetic field. Since the difference in the thermal conductivity with and without magnetic field is essential for the paramagnetic determination of the oxygen concentration, changes in the thermal conductivity due to the presence of quantities of hydrocarbons have only a nonessential influence on the determination of the oxygen concentration in the breathing gas mixture. The presence of quantities of additional substances, classes of substances or reaction products of substances in the breathing gas mixture can be inferred, on the whole, from changes in the thermal conductivity. Since at least the thermal conductivities of oxygen (O₂), moisture or water vapor (H₂O), nitrogen (N₂) and carbon dioxide (CO₂) are stored in the memory, it is advantageously possible to determine a situation in the flight operation in which a change in the thermal conductivity is present and a further component is thus present in the breathing gas mixture in addition to oxygen, nitrogen, carbon dioxide and quantities of moisture or water vapor. Even without a qualitative and/or quantitative determination of which component is present in the breathing gas mixture, this offers a great advantage for the flight operation, both in respect to an estimation of a possible health hazard for the aircraft pilot as well as to valuable information for an analysis of the state of operation of the propulsion system (turbines, engines), cooling system and other units of the aircraft during the flight operation.

The gas measuring device further has an infrared optical device with a radiation source (light source), with a detector device, with a measuring element and with a reference element. The infrared optical device is configured in combination with the control and analysis unit to carry out a measurement of an absorption of infrared radiation in the breathing gas mixture and to determine a value that indicates situations of the IR absorption in the breathing gas mixture.

Thus, there is, for example, a significant absorption of methane (CH₄) in the wavelength range around 3.1 μm, so that this wavelength range is very well suited for a measurement-based determination of methane. By contrast, carbon dioxide can be detected very well in a wavelength range around 4.26 μm and also in a wavelength range around 15 μm. A situation of the IR absorption can be determined from the ratio of the signals at the measuring element and at the reference element in respect to different gases, for example, methane or carbon dioxide, as well some other hydrocarbons, e.g., ethene, propane. Band pass filter elements tuned to the typical absorptions of the gas to be identified are preferably arranged in this case in the beam path of the IR radiation of the radiation source in front of the measuring element and the reference element.

At least one data set of data, which indicates properties of different gases, gas mixtures or vapors, is stored in the memory, At least one of the properties from the group of properties
thermal conductivity;
pressure dependence of the thermal conductivity; and
temperature dependence of the thermal conductivity is stored in the at least one data set at least for moisture contents in the breathing gas mixture and contents of the gases nitrogen, oxygen and carbon dioxide in the breathing gas mixture.

The control and analysis unit is configured to identify a special state in the breathing gas supply of the aircraft pilot, in which state at least one quantity of another gas component different from carbon dioxide, nitrogen, water vapor, moisture and oxygen is present in the breathing gas mixture, on the basis of the determined values, which indicate
moisture or a quantity of water in the breathing gas mixture,
a temperature of the breathing gas mixture,
a pressure level of the breathing gas mixture,
situations (states) of the thermal conductivity in the breathing gas mixture and
situations (states) of the IR absorption in the breathing gas mixture, and on the basis of the properties of different gases, gas mixtures or vapors, which properties are stored in the memory.

Such a special state may be classified, for example, as a status, wherein said status may describe, for example, a degree of health hazard for the aircraft pilot. The status may also describe, for example, a degree of operational safety of the breathing gas supply. The classification may be carried in a multistage form, for example, in a three-stage form (red, yellow, green) or in a five-stage form (−−, −, 0, +, ++) or be symbolized on the basis of numerical values.

The control and analysis unit is configured to provide an output signal, which indicates the special state that at least one quantity of another gas component different from carbon dioxide, nitrogen, water vapor, moisture and oxygen is present in the breathing gas mixture.

Properties of different fluids, additional gases or gas mixtures may be stored in the memory in a preferred embodiment of the monitoring system. Methane, butane, propane, ethane, butane-propane mixture, operating materials and fuels, such as kerosene, gasoline, natural gas, and liquefied gas are examples in this case.

Furthermore, operating materials, such as coolants and lubricants, as well as hydraulic fluids, lubricants or greases or anticorrosive agents are examples. The fluids, gases or gas mixtures may be based here on hydrocarbon (CₘHₙ), halogenated hydrocarbon and fluorochlorocarbon compositions. Hydraulic fluids, lubricants or greases may be based, e.g., on polymerized fluorocarbons, e.g., fluoropolymers, e.g., polytetrafluoroethylene (PTFE).

The operating materials or fuels may enter into the breathing gas supply of the airplane or aircraft possibly directly or in the form of their reaction products or vapors during the operation of the airplane or aircraft.

The potential components could be, for example, components of the exhaust gas generated during the combustion. It may be mentioned here as an example that exhaust gases and reaction products from the turbines of a jet aircraft flying in front of the aircraft in question can possibly enter as fumes into the intake manifold of the breathing gas supply unit of an aircraft flying behind it. Components of the exhaust gas are in this case, for example, unburnt kerosene, mainly carbon dioxide, and moisture as water vapor.

Comparable situations, in which unburnt fuel can enter the breathing gas supply unit, may arise during aerial refueling. Other substances, which may possibly enter into the breathing gas supply unit during the flight operation, may originate, for example, from the compressor stages of the turbines, from engines or other units. Diverse, often hydrocarbon-based cooling lubricants, as well as hydraulic fluids are usually used as fluids during the operation. In addition, various different isomers of tricresyl phosphate (TCP) may be formed, for example, under the action of heat as a reaction product of the coolant or lubricant or hydraulic fluids. TCP may present a health risk on inhalation or in case of contact with the skin or the eyes, and the effects of other chemical substances or classes of substances, which may be formed due to the heat as derivatives or reaction products during the operation of the engines, cannot therefore be considered to be absolutely harmless, either.

If various properties, especially properties which directly or indirectly represent an influence on the thermal conductivity or on the heat capacity of the substances present in the breathing gas mixture, are stored in the memory for a large number of substances, it may advantageously also be possible, in addition to the situation occurring during the flight operation, in which a change occurs in the thermal conductivity and a state with another component in addition to oxygen, nitrogen, carbon dioxide and moisture or water vapor contents in the breathing gas mixture is thus present, to estimate whether, for example, a quantity of unburnt kerosene or a quantity of reaction products of lubricant or coolant, e.g., TCP, is the cause of the change in the thermal conductivity.

An example is presented in this connection for estimating (indicating) effects of the change in the thermal conductivity under the pressure conditions prevailing at sea level corresponding to 1,013 mbar in the temperature range of about 25° C. The thermal conductivity of nitrogen equals, for example, approximately 0.0259 W·m−1·K−1. The thermal conductivity of oxygen equals, for example, approximately 0.0245 W·m−1·K−1. The thermal conductivity of carbon dioxide equals, for example, approximately 0.0164 W·m−1·K−1. The thermal conductivity of methane equals, for example, approximately 0.0337 W·m−1·K−1. Methane shall be used here as an example of a selected component for a gaseous fuel component during the operation of an aircraft.

It is seen from these data that depending on the composition of the gas mixture, the overall thermal conductivity can change with different quantities; in addition, there also are changes due to the pressure level and temperature level, which deviate from 25° C. and 1,013 mbar during the flight operation.

In another preferred embodiment, the monitoring system or the gas measuring device may have a sensor mechanism with
a velocity sensor mechanism,
an acceleration sensor mechanism or
an altitude sensor mechanism (altimeter).

The sensor mechanism may be arranged in or at the monitoring system or in or at the gas measuring device or it may be associated with the monitoring system or with the gas measuring device, for example, as an external sensor mechanism by means of a data connection. The additional sensor mechanism can advantageously be used to check the plausibility of the analyses of the monitoring system or of the gas measuring device in respect to the current operating situation of the airplane.

In another preferred embodiment of the monitoring system, additional data, which indicate additional properties of different fluids, gases, gas mixtures or vapors, which may be present as operating materials or fuels during the operation of the airplane or aircraft, may be stored in the memory. At least one of the following properties may be stored in the memory:
heat capacity;
pressure dependence of the heat capacity;
temperature dependence of the heat capacity;
density;
pressure dependence of the density;
temperature dependence of the density;
ignitability or explosiveness;
pressure dependence of the ignitability or explosiveness;
temperature dependence of the ignitability or explosiveness;
viscosity;
vapor pressure;
boiling point;
pressure dependence of the viscosity;
temperature dependence of the viscosity; and
properties that impair health, such as toxicity, carcinogenicity.

Data sheets containing a plurality of data can be, as it were, stored in this manner in the memory for different substances and reaction products, which will then make it possible, on the one hand, especially by including data of the pressure sensor and of the temperature sensor, as well as of the optional additional sensor mechanism, such as the velocity sensor mechanism, the acceleration sensor mechanism or the altitude sensor mechanism (altimeter) according to one of the above-described embodiments to check the identified special state that an additional component is present in addition to oxygen, nitrogen, carbon dioxide and moisture or water vapor contents in the breathing gas mixture in respect to the particular flight situation and to operating states of the airplane. It is also possible in this manner, for example, to also include in the analysis certain situations in the flight operation, such as full-load situation or a situation with thrust reversal of an engine and to identify it as a possible cause of the presence of foreign gas components in the breathing gas mixture in a comparison with the data stored for different substances and reaction products. For example, the coolant may in such situations reach the limits of the cooling effect and partially evaporate and also enter the breathing gas supply unit as an additional quantity of gas in possible error situations of the flight operation. Situations with partial load operation or thrust shut-off, in which possibly unburnt fuel may enter the intake manifold of the breathing gas supply unit, may be mentioned here as other examples. On the other hand, the data in the memory also make it possible to assess possible effects in respect to possible impairments to the health status of the aircraft pilot by estimating possible quantities of substances, classes of substances or reaction products in the breathing gas mixture and advantageously to expand the output signal with pertinent additional information.

An input interface or an input unit, which makes it possible to input or to select situations or states of gases, gas mixtures or vapors in the breathing gas mixture from among predefined situations or states, is provided at the monitoring system and/or in or at the gas measuring device in another preferred embodiment. The input or the selection from among predefined situations or states at the input unit may comprise the presence of at least one additional gas different from carbon dioxide, nitrogen, moisture or water vapor and/or oxygen in the breathing gas mixture of the breathing gas supply. The control and analysis unit is configured in this case to also include the selected situations or states and the data stored in the memory in the control and/or in the operation of the monitoring system during the application of upper and/or lower limit values, threshold values or alarm limits for situations that indicate the thermal conductivity and/or situations of the IR absorption to quantities in the breathing gas mixture. A specific or individual configuration can be stored in this manner at the upper and/or lower limit values, threshold values or alarm limits for the flight operation, for example, specifically for a selected aircraft or specifically for the pilot or the copilot, and an advantageous coordination of the monitoring system, adapted to the flight operation, to different types of aircraft, to different situations in the aircraft or to flight maneuvers, can thus be made possible.

An output unit is arranged at the monitoring system and/or in or at the gas measuring device in another preferred embodiment, or an output unit, which makes it possible to provide or to signal the output signal and/or states, is associated with the monitoring system and/or with the gas measuring device. The output unit may be configured, for example, to output a status signal, and such an output may be carried out acoustically, for example, in the form of a horn, or visually, for example, in the form of an LED as well as as a text message, for example, in the form of an alphanumeric display element.

The control and analysis unit is configured in another preferred embodiment to determine an oxygen concentration from the situation of the thermal conductivity in the breathing gas mixture as a function of the effect of the magnetic field and to make it available as an additional output signal.

In another preferred embodiment, the control and analysis unit is configured at the monitoring system and/or in or at the gas measuring device to determine a carbon dioxide concentration from the situation of the IR absorption in the breathing gas mixture and to make the determined carbon dioxide concentration available as an additional output signal.

Different possibilities are provided in an advantageous manner by the monitoring system according to the present invention and by the embodiments described, as well as by combinations of embodiments for determining and providing states and situations, especially situations with physical and/or chemical states during the operation of the breathing gas supply unit of an aircraft pilot. The determination of the fact that an additional gas different from oxygen, nitrogen and carbon dioxide as well as moisture or water vapor is present in the breathing gas mixture provides an important indication of the operating state of the breathing gas supply unit in the aircraft. In combination with the knowledge of the carbon dioxide concentration and of the oxygen concentration, it is also possible, in particular, to assess possible effects on the consequences for health of the dysfunctional operating state if an operating state is recognized as being dysfunctional, and these possible effects may be used to indicate to the pilot that it would be advisable to terminate the flight mission.

Embodiments were described and explained above in reference to the monitoring system and/or the gas measuring device. Another aspect of the present invention, namely, a process for monitoring a gas composition of a breathing gas supply for an aircraft pilot in airplanes or aircraft with determination of a special state of the presence of foreign gas components in the breathing gas mixture will be described below. The process according to the present invention may be carried out, for example, by a control and analysis unit, as described for the monitoring system according to the present invention and/or for the gas measuring device as well as for the embodiments of the monitoring system and/or of the gas measuring device. The following steps are carried out in a sequence of steps during the carrying out of the process according to the present invention for monitoring a gas composition of a breathing gas supply unit of an aircraft pilot in airplanes or aircraft with determination of a special state of the presence of foreign gas components in the breathing gas mixture:
detection of a current temperature level and moisture content in the breathing gas mixture;
detection of a current pressure level in the breathing gas mixture;
feeding of defined quantities of a breathing gas mixture to a thermoelectric device with cyclic magnetic field modulation;
operation of the thermoelectric device with cyclic magnetic field modulation with detection of thermovoltage signals without an influence of a magnetic field during the cyclic magnetic field modulation and
with detection of thermovoltage signals under the influence of a magnetic field during the cyclic magnetic field modulation;
signal separation of the thermovoltage signals into a d.c. voltage signal component and an a.c. voltage signal component;
feeding of defined quantities of breathing gas to an infrared optical device with a measuring element and with a reference element;
operation of the infrared optical device and detection of signals of the reference element and of the measuring element;
determination of a special state in the breathing gas supply unit of the aircraft pilot to determine whether at least one additional gas component different from carbon dioxide, nitrogen and/or oxygen as well as from moisture or water vapor is present in the breathing gas mixture,
on the basis of the current temperature level, of the moisture content and of the pressure level in the breathing gas mixture, as well as of the thermovoltage signals of the thermoelectric device and of the signals of the infrared optical device
and with inclusion of properties, e.g., thermal conductivities, pressure or temperature dependences of thermal conductivities, which are stored in a memory for different gases, gas mixtures or vapors; and
provision of an output signal, which indicates this special state in the supply of the airplane pilot with breathing gases.

Determination of a carbon dioxide concentration in the breathing gas mixture may be carried out in a preferred embodiment of the process on the basis of the signals following the detection of signals of the reference element and of the measuring element, and an output signal, which indicates the carbon dioxide concentration, may be provided.

Determination of an oxygen concentration in the breathing gas mixture on the basis of the thermovoltage signals UTH with d.c. voltage signal component UTH=and with a.c. voltage signal component UTH˜ can be carried out following the signal separation of the thermovoltage signals in a preferred embodiment of the process, and an output signal, which indicates the oxygen concentration, may be provided.

The present invention will be explained in more detail on the basis of the following description partly with reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a mask arrangement with a gas port, a connection element as well as hose lines and a measuring line according to the state of the art;

FIG. 2 is a schematic view showing a monitoring system according to the invention; and

FIG. 3 is a flow diagram with schematically shown features and showing a course of a process for monitoring a gas composition.

### DESCRIPTION OF PREFERRED EMBODIMENTS

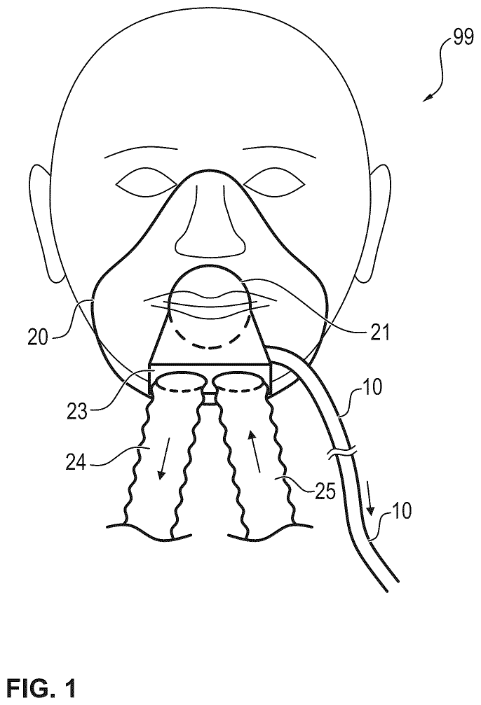

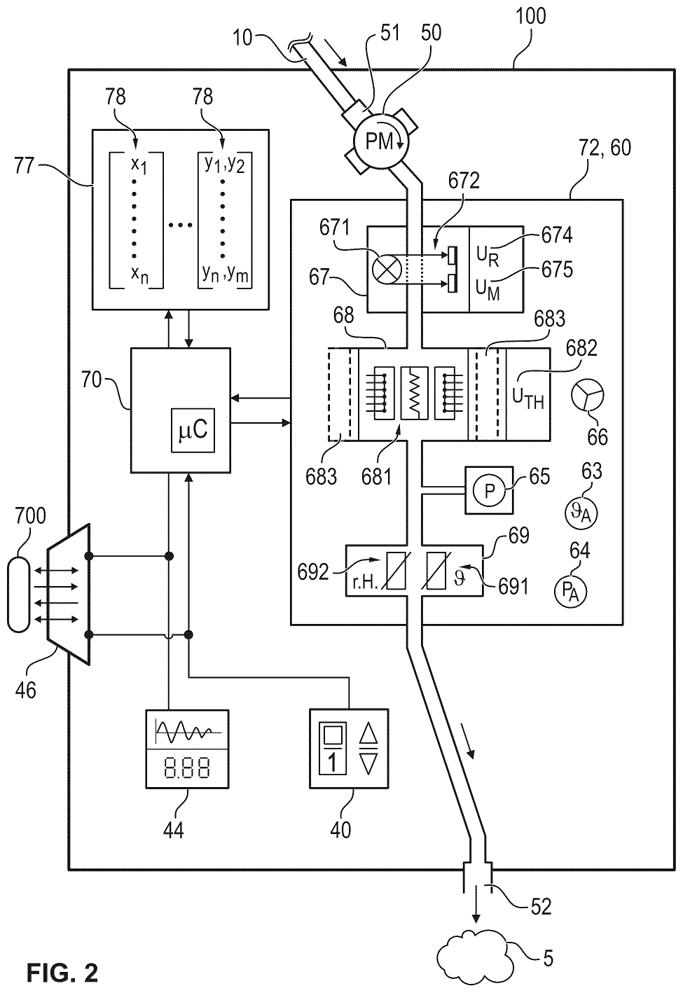

Referring to the drawings, FIG. 1 shows a schematic view of a mask arrangement with a mask 20 and a measuring line 10 according to the state of the art—according to US 20210405008 A1 (US 20210405008 A1 is incorporated herein by reference in its entirety). The mask arrangement may be used with a monitoring system 100 according to the invention as shown in FIG. 2. The monitoring system 100 is connected to a person 99 with the measuring line 10 by means of the breathing mask 20. The person 99 is an airplane pilot (pilot, copilot). The breathing mask 20 has a gas port 21, a connection element 23 as well as hose lines 24, 25.

The hose lines 24, 25 are used to remove and feed breathing gases to the person 99. The monitoring system 100 has operating elements 40, display elements 44, at least one gas delivery module 50, and a sensor mechanism 60 with at least one sensor 66. The gas delivery module 50 is preferably configured as a pump PM. In addition, the monitoring system 100 has a control and analysis unit 70. Operating elements 40, the display elements 44, the sensor mechanism 60, and the gas delivery module 50 are connected to the control and analysis unit 70 via signal and data lines or control lines, which are not shown in FIG. 2. These control lines or signal and data lines may be configured, for example, as a bus system (CAN) or network. The control and analysis unit 70 is configured and intended for controlling and/or actuating the gas delivery module 50 such that a delivery of breathing gases will take place from the breathing mask 20 through the measuring line 10 and a gas inlet 51 to the sensor mechanism 60. A quantity or partial quantity of breathing gas will thus be available for the at least one sensor 66 in the gas sensor mechanism 60 in order to detect and/or to analyze this breathing gas by measurement and to provide the result as measured values to the control and analysis unit 70. It is made possible by means of the control and analysis unit 70 to analyze, process and display the measured values on display elements 44.

FIG. 2 shows the sensor mechanism 60 of the monitoring system 100, the sensor mechanism including a gas measuring device 72, and showing details of the control and analysis unit 70, as well as an additional memory (data storage device (RAM, ROM, . . . )) 77, which is intended and is suitable for storing one or more data sets 78 with properties for moisture contents and contents of the gases nitrogen, oxygen, carbon dioxide in the breathing gas mixture and to make them available for the control and analysis unit 70. By means of delivery through the gas transport module 50, quantities of breathing gas mixture 10 reach the gas measuring device 72 via a gas inlet 51 and are again removed from the gas measuring device 72 via the gas outlet 52. Following the gas outlet 52, the quantities of breathing gas mixture 10 can be sent, for example, into the surrounding area 5, e.g., into a cockpit or into other systems. For example, chemical or physical properties, especially thermal conductivities, a pressure dependence of the thermal conductivity, as well as a temperature dependence of the thermal conductivity may be mentioned here as properties 78 stored in the memory 77 at least for moisture contents and contents of the gases nitrogen, oxygen and carbon dioxide in the breathing gas mixture as properties 78 stored in the memory 77. The control and analysis unit 70 is configured in cooperation with the gas measuring device 72 and with the data sets 78 being stored in the memory 77 to determine a special state 700—to determine whether at least one quantity of an additional gas component different from carbon dioxide, nitrogen, water vapor, moisture and oxygen is present in the breathing gas mixture—and to make this result available as an output signal 909, which indicates the special state 700, to an output unit 44 or to an optional interface 46. The gas measuring device 72 has a thermoelectric device 68, which is configured to determine a concentration of oxygen (O₂) in the breathing gas mixture by the determination of thermal conductivities in the breathing gas mixture 10. The thermoelectric device 68 has a heating and thermocouple device 681 with heating elements, thermocouples or thermopiles. A cyclic effect of a magnetic field on the heating and thermocouple device 681 is generated during the operation of the thermoelectric device 68 by means of a cooperation of the control and analysis unit and of a magnetic field device 683. The magnetic field device 693 has, in addition to the elements for guiding a magnetic field, which are schematically shown in this FIG. 2, an electromagnet with a coil array, which, just as the magnetic field itself, is not shown in this FIG. 2 for the sake of clarity. Voltage signals 682 UTH=, UTH are determined with and without influence of the magnetic field at the thermocouples or thermopiles of the heating and thermocouple device 68. The voltage signals 682 UTH=, UTH˜determined at the thermocouples or thermopiles of the heating and thermocouple device 68 indicate a thermal conductivity of the breathing gas mixture in contact with the heating and thermocouple device 681. The voltage signals 682 UTH=, UTH˜ are made available to the control and analysis unit 70. The control and analysis unit 70 is configured to determine a concentration of oxygen (O₂) from the voltage signals 682 UTH=, UTH, i.e., from a ratio of the voltage signals 682 UTH=, UTH and to make the determined concentration of oxygen (O₂) available to the output unit 44 or to an optional interface 46 and to also include the determined concentration of oxygen (O₂) in the determination of the state 700 as to whether at least one quantity of an additional gas component, different from carbon dioxide, nitrogen, water vapor, moisture and oxygen, is present in the breathing gas mixture. The gas measuring device 72 has an infrared optical device 67, which is configured for determining a concentration of carbon dioxide (CO₂) in the breathing gas mixture. The infrared optical device 67 comprises an IR radiation source 671 and a detector device 672 with a measuring element and with a reference element. Voltage signals 675 UM, 674 UR determined at the measuring element and at the reference element are made available to the control and analysis unit 70. The control and analysis unit 70 is configured to determine a concentration of carbon dioxide (CO₂) from a ratio of the voltage signals 674 UR, 675 UM and to make the determined concentration of carbon dioxide (CO₂) available to the output unit 44 or to an optional interface 46 and to also include the determined concentration of carbon dioxide (CO₂) in the determination of the state 700 as to whether at least one quantity of an additional gas component different from carbon dioxide, nitrogen, water vapor, moisture and oxygen is present in the breathing gas mixture. The gas measuring device 72 has a pressure-measuring device with a pressure sensor for a pressure measurement in the breathing gas mixture for determining a pressure level or for determining pressure changes of a pressure level in the breathing gas supply unit of the aircraft pilot. The control and analysis unit 70 is configured to determine the pressure level in the breathing gas mixture continuously from signals of the pressure sensor and to make the determined pressure level available to the output unit 44 or to an optional interface 46 and to also include the determined pressure level in the determination of the state 700 as to whether at least one quantity of an additional gas component different from carbon dioxide, nitrogen, water vapor, moisture and oxygen is present in the breathing gas mixture. The gas measuring device 72 has, in addition, a moisture and temperature measuring device 69 with a temperature sensor 691 and with a moisture sensor 692 for determining a temperature level and a moisture content in the breathing gas mixture 10, as well as changes of these parameters in the breathing gas supply unit of the aircraft pilot. The control and analysis unit 70 is configured to determine a temperature level and the moisture content in the breathing gas mixture 10 continuously from signals of the temperature sensor 691 and of the moisture sensor 692 and to make these available to the output unit 44 or to an optional interface 46 and to also include these in the determination of the state as to whether at least one quantity of a gas component different from carbon dioxide, nitrogen, water vapor, moisture and oxygen is present in the breathing gas mixture. The gas measuring device 72 may have in optional embodiments additional measuring devices for the breathing gas mixture or as an additional temperature-measuring device and/or an additional pressure measuring device with a corresponding temperature measuring device for ambient temperature 63, 64, whereby the control and analysis unit 70 is enabled to determine a temperature level and/or pressure level of the surrounding area 5 continuously and to make these available to the output unit 44 or to an optional interface 46 and to also include them in the determination of the state 700 as to whether at least one quantity of an additional gas component different from carbon dioxide, nitrogen, water vapor, moisture and oxygen is present in the breathing gas mixture 10. The gas measuring device 72 may have, for example, an acceleration sensor in optional embodiments, for example, one configured as a 2-axis or 3-axis acceleration sensor (accelerometer, a compass sensor, for example, one configured as an electronic compass, gyro compass or fluxgate compass, an altitude sensor (altimeter) or a gyro sensor (gyro meter). The control and analysis unit 70 can be enabled by means of the additional sensor mechanism 66 to also include possible effects of flight maneuvers, flight situations (take-off, landing, descent, acceleration, looping) on the breathing gas supply and in the determination of the state 700 as to whether at least one quantity of an additional gas component different from carbon dioxide, nitrogen, water vapor, moisture and oxygen is present in the breathing gas mixture 10.

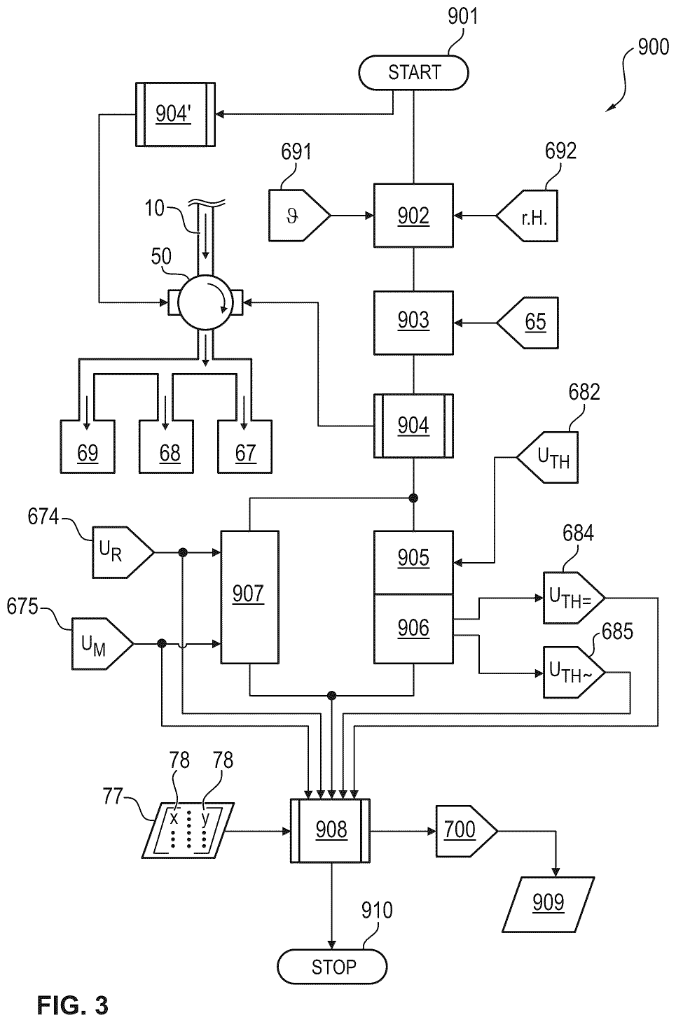

A process with a performance of a monitoring of a gas composition of a breathing gas supply of an airplane pilot in airplanes or aircraft with determination of a state 700 in which foreign gas components are present in the breathing gas mixture is shown schematically in FIG. 3 on the basis of a sequence of steps 900. A sequence of steps 900 is carried out during the performance of the monitoring from a start 901 to an end 910. A breathing gas mixture 10 being fed is available for the measurement for the moisture and temperature measuring device 69 during the start 901. Feeding of the breathing gas mixture 10 to the moisture- and temperature-measuring device 69 (FIG. 2) by means of, for example, the gas delivery module 50 for the breathing gas mixture 10 is shown only as step 904′ in order to preserve the clarity in this FIG. 3. The sequence of steps 900 may be carried out in series over time, as in the exemplary view shown in FIG. 3, and variants in which some of the steps 902 through 908 and 909 are carried out at the same time are technically possible, and they shall also be comprised by the schematic diagram according to FIG. 3. The sequence of steps 900 may be carried out once, several times cyclically or continuously

The following sequence of steps arises after a start 901:
detection 902 of a current temperature level 691 and of a moisture content (692) in the breathing gas mixture 10;
detection 903 of a current pressure level in the breathing gas mixture 10;
feeding 904 of defined quantities of breathing gas mixture 10 to a thermoelectric device 68 with cyclic magnetic field modulation by means of the gas delivery module 50;
operation 905 of the thermoelectric device 68 with cyclic magnetic field modulation with detection of thermovoltage signals UTH 682 of the thermoelectric device 68 without influence of a magnetic field during the cyclic magnetic field modulation and detection of thermovoltage signals UTH 682 of the thermoelectric device 68 under the influence of a magnetic field during the cyclic magnetic field modulation;
signal separation 906 of the thermovoltage signals UTH 682 into a d.c. voltage signal component UTH=and an a.c. voltage signal component UTH˜.
feeding 904 of defined quantities of breathing gas 10
to an infrared optical device 67 with a measuring element 675 and with a reference element 674;
operation 907 of the infrared optical device 67 with detection of signals of the reference element UR 674 and of the measuring element UM 675;
determination of a special state 700 in the breathing gas supply 10 of the airplane pilot to determine whether at least one additional gas component different from carbon dioxide, nitrogen, water vapor, moisture and/or oxygen is present in the breathing gas mixture,
on the basis of the temperature level, of the pressure level and of the moisture content in the breathing gas supply, as well as on the basis of the thermovoltage signals of the thermoelectric device 68 and of the signals of the infrared optical device 67, by including properties 78, e.g., thermal conductivities, pressure or temperature dependences of thermal conductivities, which are stored for different gases, gas mixtures or vapors in a memory 77;
provision 909 of an output signal 909, which indicates the special state 700 in the supply of the airplane pilot with breathing gases.

While specific embodiments of the invention have been shown and described in detail to illustrate the application of the principles of the invention, it will be understood that the invention may be embodied otherwise without departing from such principles.

### LIST OF REFERENCE NUMBERS

5 Surrounding area, outside air
10 Measuring line for breathing gas mixture
20 Breathing mask
21 Gas port at the breathing mask
23 Connection element
24, 25 Hose lines
40 Input unit, input interface, input elements
44 Output unit, display elements
46 Interface
50 Gas delivery module, pump P_{M}
51 Gas inlet
52 Gas outlet
60 Sensor mechanism
63 Temperature measuring device for ambient temperature
64 Pressure measuring device for the ambient pressure
65 Pressure measuring device in the breathing gas mixture
66 Additional sensors
67 Infrared optical device
671 IR radiation source
672 Detector device with measuring element and reference element
674, 675 Voltage signals UR, UM
68 Thermoelectric device
681 Heating and thermocouple device
682 Voltage signals UTH
683 Magnet device
684 D.c. voltage signal component U_{TH=}
685 A.c. voltage signal component U_{TH-}
69 Moisture and temperature measuring device in the breathing gas mixture
691 Temperature sensor
692 Moisture sensor
70 Control and analysis unit
72 Gas measuring device
77 Memory
78 Data set, data sets with properties
99 Person, pilot, airplane pilot
100 Monitoring system
700 Special state
900-910 Sequence of steps of the performance of a monitoring of a gas composition of a breathing gas supply unit

What is claimed is:

1. A monitoring system for monitoring a gas composition of a breathing gas supply of an airplane pilot in airplanes or aircraft, the monitoring system comprising:
a measuring line from a measuring location through a gas inlet at the monitoring system for a breathing gas mixture;
a gas transport module configured to feed defined quantities of the breathing gas mixture via the measuring line, from the measuring location through the gas inlet at the monitoring system;
a gas measuring device, the gas measuring device comprising:
a moisture sensor configured to determine a moisture content in the breathing gas mixture;
a pressure sensor configured to determine a pressure level in the breathing gas mixture;
a temperature sensor configured to determine a temperature of the breathing gas mixture;
a thermoelectric device comprising thermocouples or thermopiles;
an electromagnetic device comprising coils and magnetically conductive materials and arranged at the thermoelectric device;
an infrared optical device comprising a radiation source, a measuring element and a reference element;
a control and analysis unit configured to organize, to check, to control or to regulate a course of a measurement-based monitoring of the breathing gas supply, wherein the moisture sensor is configured with the control and analysis unit to determine a value that indicates a moisture content or a water content in the breathing gas mixture, wherein the pressure sensor is configured in combination with the control and analysis unit to determine a value that indicates a pressure level of the breathing gas mixture, wherein the temperature sensor is configured in combination with the control and analysis unit to determine a value, which indicates a temperature of the breathing gas mixture, wherein the electromagnetic device is configured in combination with the control and analysis unit to allow a magnetic field to act cyclically on the thermocouples or on the thermopiles, wherein the thermoelectric device is configured in combination with the control and analysis unit to carry out a determination of a thermal conductivity of the breathing gas and to determine a value that indicates situations of the thermal conductivity as a function of or without effect of the magnetic field on the quantities of breathing gas mixture fed by the gas transport module, wherein the infrared optical device is configured in combination with the control and analysis unit to carry out a measurement of an absorption of infrared radiation in the breathing gas mixture and to determine a value, which indicates situations of IR absorption in the breathing gas mixture;
a memory configured to store properties of different gases or gas mixtures, the memory being connected to the control and analysis unit, wherein at least one data set of data, which indicates properties of different gases, gas mixtures or vapors, is stored in the memory,
wherein at least one of the properties from the group of properties comprising: thermal conductivity; pressure dependence of the thermal conductivity; and temperature dependence of the thermal conductivity, is stored in the at least one data set at least for moisture contents in the breathing gas mixture and contents of the gases nitrogen, oxygen, carbon dioxide in the breathing gas mixture,
wherein the control and analysis unit is configured to identify, based on the determined values, which indicate a moisture content or a water content in the breathing gas mixture, a temperature of the breathing gas mixture, a pressure level of the breathing gas mixture, situations of the thermal conductivity in the breathing gas mixture and situations of the IR absorption in the breathing gas mixture, and based on the properties of different gases, gas mixtures or vapors, which properties are stored in the memory, a special state in the breathing gas supply of the airplane pilot, in which special state there is at least one quantity of an additional gas component different from carbon dioxide, nitrogen, water vapor, moisture or oxygen present in the breathing gas mixture, and
wherein the control and analysis unit is configured to provide an output signal which indicates the special state that at least one quantity of an additional gas component different from carbon dioxide, nitrogen, water vapor, moisture and oxygen is present in the breathing gas mixture.

2. A monitoring system in accordance with claim 1, wherein different properties of different additional fluids, gases or gas mixtures are stored in the memory.

3. A monitoring system in accordance with claim 1, further comprising
a velocity sensor mechanism;
an acceleration sensor mechanism; or
an altitude sensor mechanism.

4. A monitoring system in accordance with claim 1, wherein additional data are stored in the memory, which indicate different properties of different fluids, gases, gas mixtures or vapors, the additional data comprising at least one of the following properties:
heat capacity;
pressure dependence of the heat capacity;
temperature dependence of the heat capacity;
density;
pressure dependence of the density;
temperature dependence of the density;
ignitability or explosiveness;
pressure dependence of the ignitability or explosiveness;
temperature dependence of the ignitability or explosiveness;
viscosity;
vapor pressure;
boiling point;
pressure dependence of the viscosity;
temperature dependence of the viscosity;
properties that are harmful for health including toxicity and or carcinogenicity.

5. A monitoring system in accordance with claim 1, further comprising an input interface or an input unit configured to input or select from among predefined situations or states of gases, gas mixtures or vapors in the breathing gas mixture,
wherein an input or a selection from among predefined situations or states at the input unit comprises a presence of at least one additional gas different from carbon dioxide, nitrogen, water vapor and/or oxygen in the gas mixture of the breathing gas supply, and
wherein the control and analysis unit is configured to include the selected situations or states and the data stored in the memory in controlling and/or operating the monitoring system during the application of upper and/or lower limit values, threshold values or alarm limits for situations that indicate the thermal conductivity and/or for situations of the IR absorption to quantities in the breathing gas mixture.

6. A monitoring system in accordance with claim 1, further comprising an output unit configured to provide or to indicate the output signal and/or the states.

7. A monitoring system in accordance with claim 1,
wherein the control and analysis unit is configured to determine an oxygen concentration as a function of the effect of the magnetic field from the situation of the thermal conductivity in the breathing gas mixture and to make the determines oxygen concentration available as an additional output signal; and/or
wherein the control and analysis unit is configured to determine a carbon dioxide concentration from the situation of the IR absorption in the breathing gas mixture and to make the situation of the IR absorption available as an additional output signal.

8. A process for monitoring a gas composition of a pilot's breathing gas supply of an airplane pilot in airplanes or aircraft with determination of a special state in which foreign gas components are present in a pilot's breathing gas mixture of the pilot's breathing gas supply, the process comprising the steps of:
detecting a current temperature level and a current moisture content in the pilot's breathing gas mixture;
detecting a current pressure level in the pilot's breathing gas mixture;
feeding defined quantities of the pilot's breathing gas mixture to a thermoelectric device with cyclic magnetic field modulation;
operating the thermoelectric device with cyclic magnetic field modulation with detection of thermovoltage signals without an influence of a magnetic field and under an influence of a magnetic field during the cyclic magnetic field modulation;
separating a d.c. signal component and an a.c. voltage signal component of the thermovoltage signals;
feeding defined quantities of the pilot's breathing gas mixture to an infrared optical device comprising a measuring element and a reference element;
operating the infrared optical device and detecting signals of the reference element and of the measuring element;
determining a special state in the breathing gas supply of the airplane pilot to determine whether at least one additional gas component different from carbon dioxide, nitrogen and/or oxygen is present in the pilot's breathing gas mixture based on the current temperature level, the moisture content and of the pressure level in the pilot's breathing gas mixture, the thermovoltage signals of the thermoelectric device and the signals of the infrared optical device and based on properties stored in a memory for different gases, gas mixtures or vapors including thermal conductivities, pressure or temperature dependences on thermal conductivities; and
providing an output signal, which indicates the special state in the pilot's breathing gas mixture of the pilot's breathing gas supply.

9. A process in accordance with claim 8, further comprising:
determining a carbon dioxide concentration in the pilot's breathing gas mixture based on signals of the detection element and of the reference element and of the measuring element, and
providing an output sign al which indicates the carbon dioxide concentration.

10. A process in accordance with claim 9, further comprising:
determining an oxygen concentration in the pilot's breathing gas mixture based on the thermovoltage signals with d.c. voltage signal component and a.c. voltage signal component following the signal separation of the thermovoltage signals; and
providing an output signal which indicates the oxygen concentration.
